(19) United States
(12) Patent Application Publication
(10) Pub. No.: US 2026/0137952 A1
(43) Pub. Date: May 21, 2026

(54) THERAPY LIGHT

(71) Applicants: Ashley Fernandez, Kitchener (CA); Cindy Prince Briggs, Kitchener (CA)

(72) Inventors: Ashley Fernandez, Kitchener (CA); Cindy Prince Briggs, Kitchener (CA)

(21) Appl. No.: 18/953,268

(22) Filed: Nov. 20, 2024

(51) Int. Cl. A61N 5/06

(52) U.S. Cl. CPC A61N 5/0618; A61N 2005/0651; A61N 2005/0663

(57) ABSTRACT

A therapy light that can be utilized for therapy and play to provide a sensory interaction for the user to enhance visual stimulation. The light includes a first tube member having a first end and a second end having a hollow passage therebetween. A plurality of light members are disposed within the hollow passage of the first tube member and are configured to emit light upon movement of the invention. The first tube member has secured to the outer wall thereof a diffraction member. The diffraction member includes an outer surface having light diffractors formed thereon having a multitude of angled surfaces to direct light emissions in a multitude of directions. The first tube member is disposed within a second tube member wherein the second tube member is transparent and having a closed first end and second end with hollow passage to accommodate the first tube member.

### FIELD OF THE INVENTION

The present invention relates generally to light devices, more specifically but not by way of limitation, a light emitting device that is configured to be utilized for play and sensory interaction wherein the present invention includes light member that are motion activated and the light device provides diffraction of the emitted light.

### BACKGROUND

Light therapy and the use of lights for treating various mental conditions as well as provide visual and sensory interaction has been increasing in use over the last decade. Maladies such as but not limited to seasonal affective depression, autism and other illnesses can be treated in part through the utilization of light therapy. For both seasonal and non-seasonal depression, the effectiveness of light therapy is approximately the same as antidepressant medications, or popular forms of psychotherapy such as cognitive behavioral therapy. Evidence from some studies suggest, light therapy improves symptoms in up to sixty percent of people. Seasonal affective depression is triggered by a drop in daily sunlight exposure. Light affects complex systems that govern the twenty-four-hour circadian clock in the brain, which regulates not just our sleep and wake cycles but also digestion, hormonal activity, and other important bodily functions. In recent years, scientists have discovered additional nerve pathways from light receptors in the retina to other parts of the brain. This includes the prefrontal cortex, which is involved in regulating mood and cognition.

Sensory integration therapy integrates touch, smell, taste, sight, and hearing to support individuals with sensory processing challenges, such as those faced by children on the autism spectrum. These therapy techniques target the unique ways each child experiences the world. Through utilization of light therapy, therapists help children navigate and interpret the sensory information that can often be overwhelming or confusing. Within the therapeutic setting, sensory lights are actively utilized as part of the therapy session. By incorporating these lights into sensory integration therapy, therapists can create a range of experiences from calming to engaging. This versatility is particularly valuable in addressing the individual needs of children with autism, who may have diverse and fluctuating sensory preferences. Light therapy aims to bridge gaps in sensory interaction by providing universally accepted devices that enhance visual stimulation and promote inclusivity.

Accordingly, there is a need for a light device that can be utilized for alternate reasons and/or techniques that is configured to provide motion activated lights in an assembly that can be easily engaged by an individual.

### SUMMARY OF THE INVENTION

It is the object of the present invention to provide a light emitting device that is configured to broadcast light upon motion of the device wherein the present invention includes a first tube member.

Another object of the present invention is to provide a light device that can be utilized for play or therapy wherein the first tube member includes a first end and a second end having a hollow passage therebetween.

A further object of the present invention is to provide a light emitting device that is configured to broadcast light upon motion of the device wherein the first tube member includes a plurality of light members disposed in the hollow passage thereof.

Yet a further object of the present invention is to provide a light device that can be utilized for play or therapy wherein the present invention includes a second tube member having a first end and a second end.

Still another object of the present invention is to provide a light emitting device that is configured to broadcast light upon motion of the device wherein the second tube member includes a hollow passage intermediate the first end and second end.

An additional object of the present invention is to provide a light device that can be utilized for play or therapy wherein the second tube member has a diameter larger than that of the diameter of the first tube member so as to receive the first tube therein.

Yet a further object of the present invention is to provide a light emitting device that is configured to broadcast light upon motion of the device wherein the present invention further includes a diffraction member wherein the diffraction member is planar in manner and flexible.

Still another object of the present invention is to provide a light device that can be utilized for play or therapy wherein the diffraction member is circumferentially disposed around the exterior of the first tube member and is configured to diffract light emitting outwards from the first tube member.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a perspective view of an exemplary light member of the present invention; and

FIG. 2 is a perspective view of the first tube member having light members disposed therein; and

FIG. 3 is a top view of the diffraction member; and

FIG. 4 is a perspective view of the present invention in an assembled state.

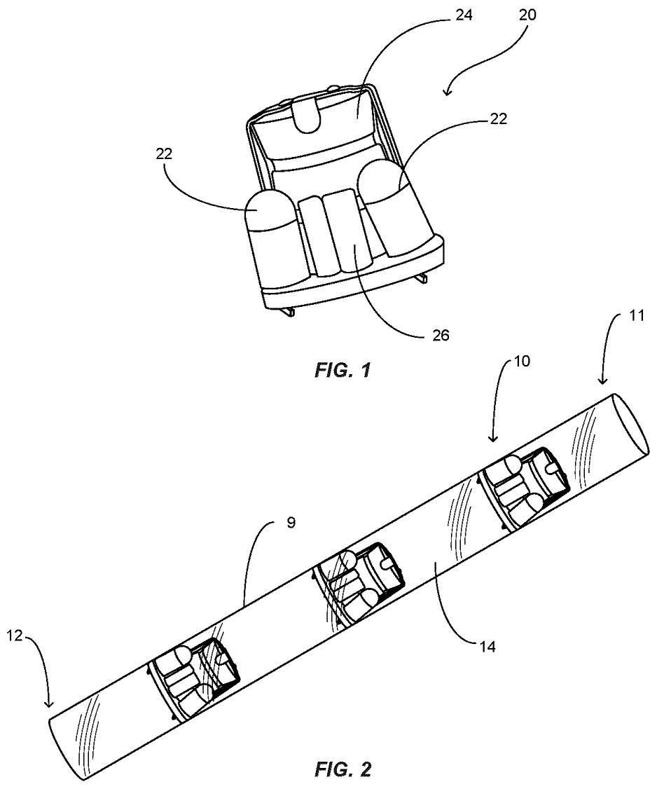

### DETAILED DESCRIPTION

Referring now to the drawings submitted herewith, wherein various elements depicted therein are not necessarily drawn to scale and wherein through the views and figures like elements are referenced with identical reference numerals, there is illustrated a therapy light 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the therapy light 100 should be understood to be utilized for scenarios such as but not limited to play and/or therapeutic methodologies. The therapy light 100 includes a first tube member 10 that is cylindrical and elongated in shape. The first tube member 10 includes a first end 11 and a second end 12. A hollow passage 14 is present intermediate the first end 11 and second end 12. Diameter of the first tube member 10 can vary but is sufficient in measurement to accommodate a plurality of light members 20 therein. The first tube member 10 includes wall member 9 wherein the wall member 9 is manufactured from a transparent material such as but not limited to plastic. It should be understood within the scope of the present invention that the first tube member 10 could be manufactured in various alternate lengths and sizes so as to be releasably secured within the second tube member 30 as is further discussed herein.

The light member 20 is releasably secured within the first tube member 10 utilizing suitable durable techniques. Light member 20 incudes LED members 22 that are configured to emit light therefrom. It should be understood within the scope of the present invention that the light member 20 could be configured to emit a variety of colors. The light member 20 includes switch member 24. Switch member 24 provides operation of the LED members 22 being operably coupled thereto. The switch member 24 in a preferred embodiment is a conventional electronic tilt switch that provides closing of the circuit needed to complete power transmission to the LED members 22. Utilization of a switch such as but not limited to a tilt switch provides a motion activated operation which is desired in the preferred embodiment of the present invention. The light member 20 includes a conventional power supply 26 providing direct current power for operation of the light member 20. While three light members 20 are illustrated disposed within the first tube member 10 herein, it should be understood within the scope of the present invention that as few as one light member 20 could be disposed within the hollow passage 14 of the first tube member 10 or more than three.

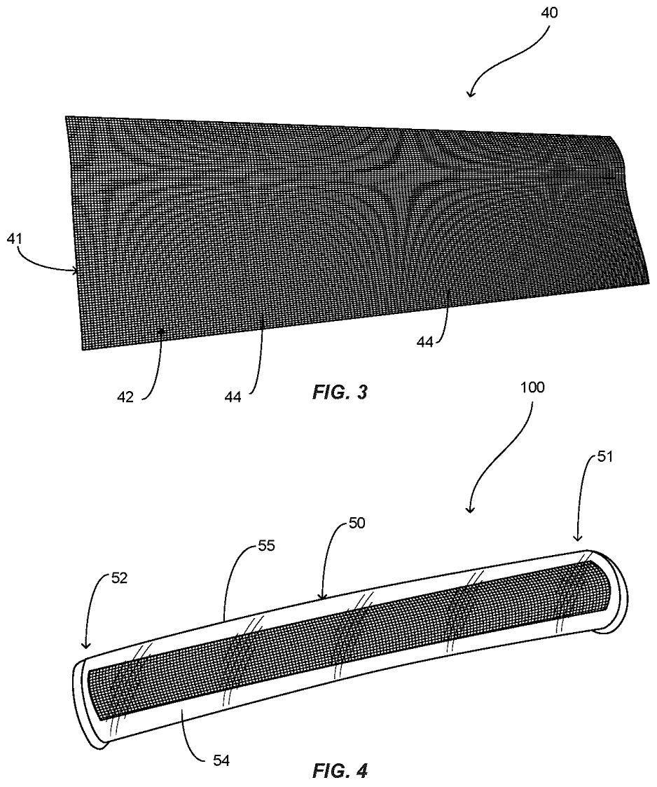

Referring in particular to FIG. 3, the diffraction member 40 is illustrated therein. The diffraction member 40 is planar in manner having a first side 41 and a second side 42. The diffraction member 40 is manufactured from a flexible material such as but not limited to plastic sheeting. The diffraction member 40 is configured to be circumferentially disposed around wall 9 of the first tube member 10. The second side 42 of the diffraction member 40 is textured with light diffractors 44 which are manufactured to have a plurality of angled surfaces so as to diffract light emitting therethrough. The first side 41 has disposed thereon a pressure sensitive adhesive to facilitate securing to the wall 9 of the first tube member 10.

The therapy light 100 includes a second tube member 50 being similarly shaped as the first tube member 10. The second tube member 50 includes wall 55 manufactured from a transparent material such as but not limited to plastic. The second tube member 50 is elongated and cylindrical in shape have a first end 51 and a second end 52 with hollow passage 54 being intermediate thereto. The second tube member 50 is of sufficient diameter to permit the securing of the first tube member 10 therein as is illustrated herein in FIG. 4. The second tube member 50 houses the first tube member 10 in order to provide impact protection for the first tube member 10 that has had the diffraction member 40 mounted thereto. It should be understood within the scope of the present invention that the second tube member 50 could be manufactured in alternate diameters and lengths.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A light configured to be utilized for play or therapy to provide visual stimulation wherein the light comprises:
a first tube member, said first tube member having a first end and a second end, said first tube member having a hollow passage between said first end and said second end, said first tube member having a wall member, said first tube member being manufactured from a transparent material;
at least one light member, said at least one light member being disposed within said hollow passage of said first tube member, said at least one light member configured to emit light ensuing movement of the first tube member;
a second tube member, said second tube member having a first end and a second end, said second tube member having a hollow passage between said first end and said second end, said second tube member having a diameter that is great than that of a diameter of the first tube member; and
wherein said first tube member is disposed within said hollow passage of said second tube member.

2. The light configured to be utilized for play or therapy to provide visual stimulation as recited in claim 1, wherein said second tube is manufactured from a transparent material.

3. The light configured to be utilized for play or therapy to provide visual stimulation as recited in claim 2, and further including a diffraction member, said diffraction member having a first side and a second side, said diffraction member being circumferentially disposed around the wall member of said first tube member.

4. The light configured to be utilized for play or therapy to provide visual stimulation as recited in claim 3, wherein said first side of said diffraction member is adjacent said wall member of said first tube member.

5. The light configured to be utilized for play or therapy to provide visual stimulation as recited in claim 4, wherein said second side of said diffraction member has a multitude of light diffractors formed thereon.

6. The light configured to be utilized for play or therapy to provide visual stimulation as recited in claim 5, wherein said light diffractors have a multitude of angled surfaces.
